(19) United States
(12) Patent Application Publication
(10) Pub. No.: US 2022/0388737 A1
(43) Pub. Date: Dec. 8, 2022

(54) KIT FOR OPHTHALMIC SOLUTIONS IN SCREW-CAP DROPPER BOTTLES

(71) Applicant: Alejandro GAMBOA BURGOS, Lima (PE)

(72) Inventor: Alejandro GAMBOA BURGOS, Lima (PE)

(21) Appl. No.: 17/774,731

(22) PCT Filed: Nov. 5, 2019
(86) PCT No.: PCT/PE2019/050005

§ 371 Date: May 5, 2022

(51) Int. Cl. B65D 47/18; B65D 41/04; A61F 9/00

(52) U.S. Cl. CPC B65D 47/18; B65D 41/0435; A61F 9/0008; B65D 2251/02

(57) ABSTRACT

The present invention relates to a kit for ophthalmic solutions contained in screw-cap dropper bottles, which comprises a fastening element and an applicator device for solutions, where the fastening element includes a manual gripping section followed by a cup-shaped cavity suitable for housing the screw-cap of the dropper bottle under pressure; such fastening element has the ability to serve as an extension in order to cap or uncap the screw-cap of the dropper bottle with ease, in addition to keeping such cap coupled to the fastening element while the bottle is in use.

### TECHNICAL FIELD

The present invention falls within the technical field of ophthalmic product dispensers, specifically an ophthalmic kit applicator for solutions contained in screw-cap dropper bottles is unveiled.

### STATE OF THE ART

In the state of the art there are some tools and/or accessories for applying ophthalmic solutions that come in dropper bottles with caps. However, solution applicator devices in multi-drop bottles which have a means to guide in a simple way the holder of the applicator device to a certain distance and position of the eye—are designed to be fixed to the dropper bottle for as long as its contents last, making it difficult to cap and uncap the dropper bottle without removing the applicator device, due to the fact that its holder structure obstructs or greatly reduces the possibilities of capping and uncapping the bottle.

Through the technical document of patent PCT/PE2018/000022, an applicator for ophthalmic substances for single-dose containers is made known, which comprises oval lips at a suitable level to be placed in contact with the sections of skin adjacent to the eye; such holder structure which helps position the bottle at a certain distance and position, nonetheless, limits the ease of the action of capping and uncapping the dropper bottle—which requires to be capped again to avoid any risk of contamination of the ophthalmic substance. Furthermore, this precedent is limited to only single-dose dropper bottles.

Likewise, in the state of the art there is the document of patent US20050101921A1, published on May 12, 2005, entitled “Dropper positioning device”, which refers to a device that helps to apply drops in the eyes from a bottle, preferably single-dose containers. This invention comprises an eye guide which is preferably oval-shaped, a substantially rigid extension portion joined to the eye guide and a bottle connector portion coupled at a second end of the extension portion; said bottle connector portion further consists of a base portion joined to the rigid extension portion, said base portion includes a slot that is sized to receive a single-use disposable bottle, a flat handle portion and also having a pair of compression tabs located on opposite sides of the slot. In a preferred embodiment, said invention comprises a positioning indicator for proper placement of a disposable bottle having a flat handle portion.

Another previous document is patent US20080208148A1, published on May 25, 2005, entitled “Ophthalmic Device”, which reveals an ophthalmic device to assist in securing the precise dispensing of an ophthalmic substance to an eye; said device comprising an elastic oval border having an open end and an opening at the opposite end. The periphery of the open end is adapted to receive and secure the nozzle associated with an eye drop dispenser. The opening may take the form of an asterisk so that the opening is able to fit and secure an eye drop container. In FIG. 2 of said antecedent it is observed that, from a frontal view, the oval border has concave lips to the structure in order to be adapted ergonomically into the eye.

Another preceding document is U.S. Pat. No. 4,960,407A, published May 26, 1989, entitled “Disposable eye drop dispensing instrument for post-surgical and general use”, which comprises a generally oval or periorbital ring contoured to fit within the orbital or periorbital area of the body of a patient and at least one column extending from the oval ring usually perpendicular to a plane of the oval ring, the post joined to the neck and shoulder region of the bottle. In a preferred form at least one column extends to the rear of the container where a cradle base will be located.

Likewise, in the state of the art there is the antecedent patent WO2004043315A, published on May 27, 2004, entitled “A device and method to facilitate the application of liquid material to the eye”, which refers to a device that facilitates the dispensing of liquid material to the eye, which has an outlet opening configured so as to substantially prevent liquid droplets from adhering thereto, the device has a support mechanism that can be reclined on an eyelid and can be used to evert such eyelid; when a liquid dispenser is placed, then the support mechanism, the eye drops, or any other liquid to be applied to one eye, can be performed in an optimal position on said eye from the liquid dispenser outlet opening, thus allowing an easy one-handed application. This invention also relates to a liquid dispenser having an integral support for adjoining against and everting an eyelid. In a preferred form, the support material may be of a substantially elastic material.

Nonetheless, in the state of the art, it has not been found a single applicator kit for ophthalmic substances in dropper bottles with a screw-cap, which integrates a set of advantageous technical characteristics, with extremely useful benefits for the user, such as simplifying the capping and uncapping action through an extension, wherein said extension has the ability to hold the cap coupled to the bottle while it is in use. In addition, it allows a positioning of the bottle in the eye at an angle of 30° to 60° with respect to a horizontal plane, which simplifies the application of the ophthalmic solution, providing open spaces in the structure to insert, remove and press the container directly with the fingers, and substantially allowing to view the contents and the label of the dropper bottle, among other advantages considered important in this proposed invention.

### DESCRIPTION OF THE INVENTION

The present invention refers to a kit for ophthalmic solutions in dropper bottles, or droppers, with a screw-cap which comprises:
a fastening element in the form of an elongated and narrow bar, wherein this fastening element includes: a manual gripping section (handle) followed by a cup-shaped cavity suitable for housing the cap of the dropper bottle by pressure; in a preferred way, this cup-shaped cavity has a grooved inner surface; this fastening element has the ability to serve as an extension to easily cap or uncap the dropper bottle cap, in addition to keeping the bottle cap coupled to the fastening element while the bottle is in use.
an ophthalmic solution applicator, which includes: ring-shaped portion for attaching with grooved inner surface (or other similar shaped portion for attaching) suitable for holding is the neck of the dropper bottle; allowing free mobility, that is, without touching the cap of the dropper bottle; a solid structure with two or more extensions or side walls arranged around the fixation portion; an oval border arranged at one end of the two or more extensions of the solid structure, the oval border has concave upper and lower lips with a height difference between them in order to facilitate the positioning of the applicator at an angle between 30 and 60° with respect to a horizontal plane.

In one embodiment, a through hole is provided between the handgrip portion and the cup-shaped cavity of the fastening element, that goes through the smaller end of the cup-shaped cavity, allowing the cap to be removed from the fastening element in case of jamming.

The oval border of the applicator located at one end of the solid structure, with its specific shape and position with respect to the eye, facilitates the positioning of the applicator at an angle between 30° and 60°, preferably 45°, with respect to a horizontal plane. Preferably a guide protrusion sticking out from the oval border is provided which serves as a guide to hold open the lower lid of the eye.

The applicator device is designed to remain coupled to the dropper bottle until the contents of said dropper bottle last. In another preferred form, the fixation portion comprises a limiting protrusion (grooves) that works as a cap mechanism to insert the single-dose container to a certain point and lock its movement.

The oval border, which ergonomically fits the curvature of the eye, firmly holds the lower and upper eyelids, preventing blinking during the dosing of the drop in the eye, and allowing the drop to be applied to the eye with total safety and confidence, nullifying the dosing outside the eye which could be caused by tremors or inexperience of the patient.

### SHORT DESCRIPTION OF THE ILLUSTRATIONS

The following figures illustrate some embodiments of the present invention, said figures is or their description are not intended to limit in any way the scope of protection defined by the claims.

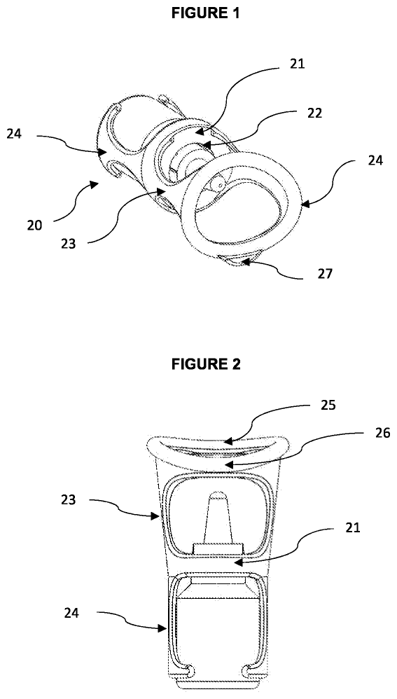

FIG. 1: A perspective view of the applicator for ophthalmic solutions is shown.

FIG. 2: A front view of the applicator for ophthalmic solutions is shown.

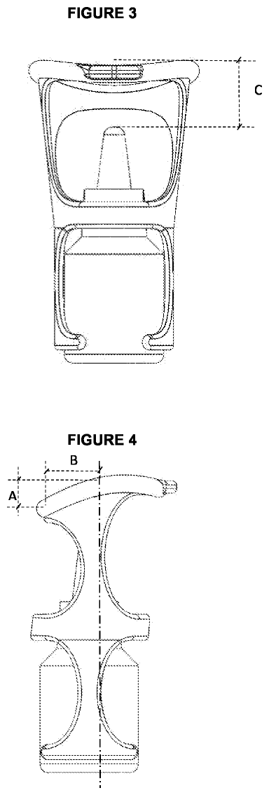

FIG. 3: A rear view of the ophthalmic solutions applicator is shown.

FIG. 4: A side view of the ophthalmic solutions applicator is shown.

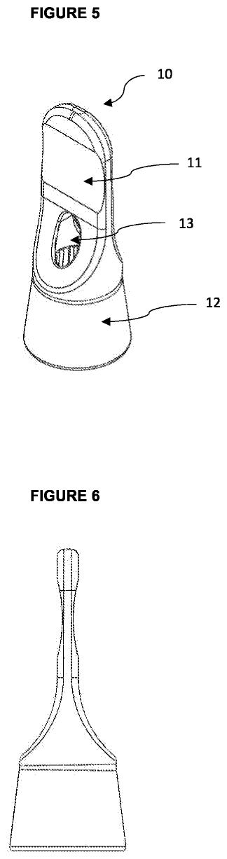

FIG. 5: A perspective view of a first embodiment of the fastening element is shown.

FIG. 6: A side view of the first embodiment of the fastening element is shown.

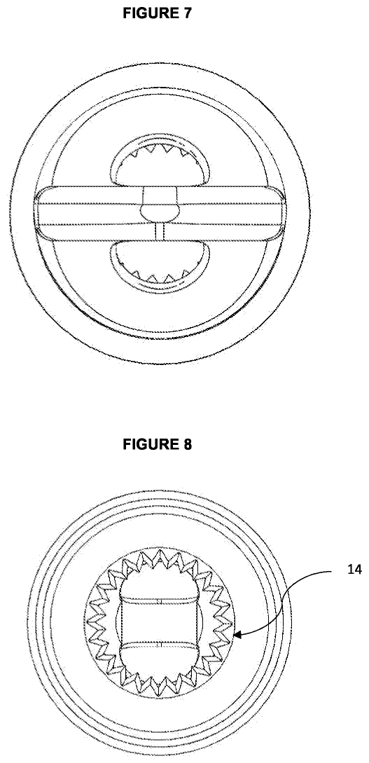

FIG. 7: A top view of the first embodiment of the fastening element is shown.

FIG. 8: A bottom view of the first embodiment of the fastening element is shown.

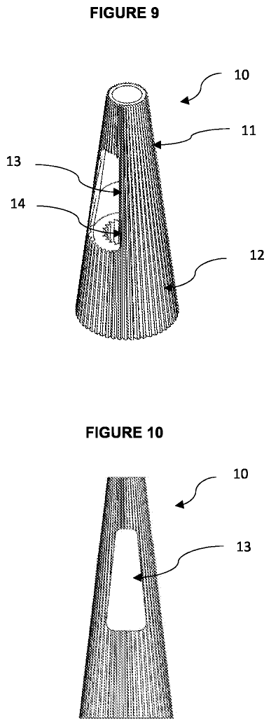

FIG. 9: A perspective view of a second embodiment of the fastening element is shown.

FIG. 10: A front view of the second embodiment of the fastening element is shown.

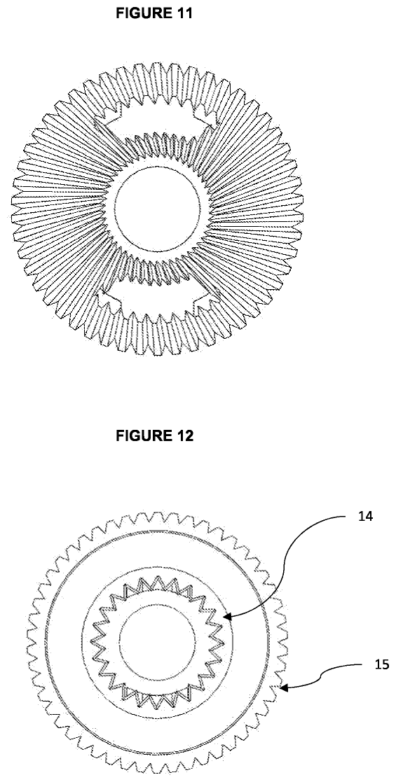

FIG. 11: A top view of the second embodiment of the fastening element is shown.

FIG. 12: A bottom view of the second embodiment of the fastening element is shown.

### PREFERRED EMBODIMENT OF THE INVENTION (DETAILED DESCRIPTION OF THE INVENTION)

FIG. 1 shows the applicator for ophthalmic solutions (20) including ring-shaped portion for attaching (21) with preferably a grooved inner surface (22) suitable for holding the neck of a dropper bottle, leaving free mobility for the cap of the dropper bottle; a solid structure (23) with two or more extensions or side walls arranged around the fixation portion; an oval border (24) arranged at one end of the two or more extensions of the solid structure, the oval border has concave upper lip (25) and concave lower lip (26) with a height difference between them in order to facilitate the positioning of the applicator at an angle between 30 and 60° with respect to a horizontal plane. In this figure you can see the guiding protrusion (27) and the way in which the solid structure extends, leaving the fixation portion in an intermediate zone.

FIGS. 2, 3 and 4 explain the assembly detail of the applicator for ophthalmic solutions seen from other angles, it can be seen that the distance from the highest point of the upper lip (25) to the most extreme and highest point of the lower lip (26), seen from a side view, i.e., distance A, is between 4 to 6 mm; the vertical distance from the tip of the dropper to the horizontal projection of the highest point of the ophthalmic solutions applicator, i.e., distance C is between 13 and 15 mm. And from a side view, the horizontal distance from the tip of the lower lip to the center, i.e., the distance B is between 12 and 14 mm. These distances have been obtained through a trial and error process to allow ophthalmic substances to enter the eye in an eye-centered position.

Likewise, in a preferred embodiment, the fixation portion is an opening with a plurality of flexible flippers (fins) around it that converge at a central point.

In a preferred form, the solid structure has two side walls, which leave a substantially open space at the front and rear of the applicator device.

FIGS. 5, 6, 7 and 8 show a first embodiment of the fastening element (10), where the manual gripping section (11) has a substantially flat shape, with a smooth dent to facilitate manual gripping by the user and not allowing his fingers to slip easily. The through hole (13) is also observed

FIGS. 9, 10, 11 and 12 show a second embodiment of the fastening element. In this embodiment, the cup-shaped cavity (12) is a hollow conical section to hold the cap of the bottle under pressure as it is inserted, and may include inside some elements to help fasten it down by pressure, such as a grooved inner surface (14), a multipurpose glue or adhesive element, among other similar fastening aid elements. The gripping section of the fastening element could be an extension of the conical section of the cup-shaped cavity, with a grooved external surface (15).

It should be noted that the term “cup”, as it is used herein, encompasses a large number of shapes of internal or external walls, such as conical, cylindrical, hexagonal, hemispherical hollow sections, etc.

The fastening element (10) has a height greater than the separation span between the fixation portion (21) and the oval border (24) of the ophthalmic solution applicator, in such a way that it allows easy gripping by the handle of the fastening element when the latter is used to remove the cap of the dropper bottle.

In another preferred form, the applicator device and the fastening element are made of a bacteriostatic or bactericidal material, such as, for example, a plastic material containing silver ions.

In another preferred form, a border of a flexible material such as rubber, silicone, soft plastic, or the like, is coupled to the oval border to soften the positioning of the device as it contacts the periphery of the eye.

1. (canceled)

2. (canceled)

3. (canceled)

4. (canceled)

5. (canceled)

6. (canceled)

7. (canceled)

8. (canceled)

9. (canceled)

10. A kit for ophthalmic solutions contained in dropper bottles with a screw-cap, characterized in that it comprises:
a fastening element (10), wherein said fastening element includes: a manual gripping section (11) followed by a cup-shaped cavity (12), suitable for holding the cap of the dropper bottle by pressure, wherein a through hole (13) is provided between the handgrip portion and the cup-shaped cavity, that goes through the smaller end of the cup-shaped cavity.
an ophthalmic solution applicator (20), which includes: ring-shaped portion for attaching (21) with a grooved inner surface (22) suitable for holding the neck of the dropper bottle; allowing free mobility for the cap of the dropper bottle; a solid structure (23) with two or more extensions or side walls arranged around the fixation portion; an oval border (24) arranged at one end of the two or more extensions of the solid structure, the oval border has a concave upper lip (25) and a concave lower lip (26) with a height difference between them to facilitate the positioning of the applicator at an angle between 30 and 60° with respect to a horizontal plane

11. The kit for ophthalmic solutions contained in dropper bottles with screw-cap, according to claims 1, characterized in that the cup-shaped cavity is a hollow conical section.

12. The kit for ophthalmic solutions contained in dropper bottles with screw-cap, according to claim 1, characterized in that the manual gripping portion is a flat plate.

13. The kit for ophthalmic solutions contained in dropper bottles with screw-cap, according to claim 1, characterized in that a protruding guide elongation (27) is located on the upper lip (25) of the oval border that serves as a guide.

14. The kit for ophthalmic solutions contained in dropper bottles with screw-cap, according to claim 1, characterized in that it is made of a bacteriostatic or bactericidal material.

15. The kit for ophthalmic solutions contained in dropper bottles with screw-cap, according to claim 14, characterized in that the bacteriostatic material comprises silver ions.

16. The kit for ophthalmic solutions contained in dropper bottles with screw-cap, according to claim 1, characterized in that the oval border comprises a ring of a flexible.
